(19) United States
(12) Patent Application Publication
(10) Pub. No.: US 2014/0221911 A1
(43) Pub. Date: Aug. 7, 2014

(54) CLOT DETECTION BASED ON SIGNAL-TIME HISTORY DIAGNOSTICS

(71) Applicant: Edwards Lifesciences Corporation, Irvine, CA (US)

(72) Inventors: Paul S. Van Wieren, Costa Mesa, CA (US); Michael Higgins, Huntington Beach, CA (US)

(73) Assignee: Edwards Lifesciences Corporation, Irvine, CA (US)

(21) Appl. No.: 14/165,297

(22) Filed: Jan. 27, 2014

Related U.S. Application Data
(60) Provisional application No. 61/760,007, filed on Feb. 1, 2013.

(51) Int. Cl. A61M 5/172; A61M 5/168

(52) U.S. Cl. CPC A61M 5/1723; A61M 5/16854
USPC 604/65

(57) ABSTRACT

The present disclosure provides a sensor system that includes a blood access device, a flow controller, and an analyte sensor. The system is configured to flush or draw a volume of fluid through the blood access device at least once; determine a signal-time history corresponding to signals received from the sensor; determine a duration of a portion of the signal-time history; determine whether the duration is greater than a predetermined threshold value; and detect the occlusion in the blood access device when the duration is greater than the predetermined threshold value.

### CROSS-REFERENCE TO RELATED APPLICATIONS

This application claims benefit of U.S. Provisional Application No. 61/760,007, filed Feb. 1, 2013, which is incorporated by reference herein.

### BACKGROUND

Some analyte monitoring systems use cyclical draw and flush routines to alternately draw fluid up to a sensor assembly and then flush calibration solution over the sensor assembly. In some instances, the catheter or sensor becomes occluded and prevents an effective draw from occurring. This occlusion causes the system to fail to draw all or some of the blood that is intended for sampling during the next draw phase. As a result, the sensor can be continuously bathed in calibration solution or the blood draw can be diluted with the calibration resulting in inaccurate measurements.

### BRIEF SUMMARY

In a first aspect, a system for detecting an occlusion in a blood access device is provided. In some embodiments, the system includes a blood access device in fluid communication with a calibrant solution source and a circulatory system of a patient; a flow controller configured to sequentially draw blood from the circulatory system and flush fluid from the calibrant solution source through the blood access device, the sequential draw and flush defining a draw and flush cycle; and a sensor coupled to the blood access device and configured to generate one or more portions of a signal-time history associated with the draw and flush cycle. The system also includes a memory; a processor; and a computing module, stored in the memory, executable by the processor, and configured to cause the processor to perform a series of actions. The actions include operating the flow controller to sequentially draw blood and flush fluid through the blood access device; determining a waveform for the draw and flush cycle from the sensor; determining a time duration of a predetermined portion of the signal-time history associated with the draw and flush cycle; determining whether the time duration is greater than a predetermined threshold value; and detecting an occlusion in the blood access device when the time duration of the signal-time history is greater than the predetermined threshold value.

In a further embodiment, the sensor is a glucose sensor. In some embodiments, the system also halts display of information on a display when the occlusion is detected. In still further embodiments, the predetermined threshold value is determined based on a standard signal-time history analyzed when an occlusion is not present.

In another aspect, a method for detecting an occlusion in a blood access device is provided. In some embodiments, the method includes providing a blood access device in fluid communication with a calibrant solution source and a circulatory system of a patient; providing a flow controller configured to sequentially draw blood from the circulatory system and flush fluid from the calibrant solution source through the blood access device, the sequential draw and flush defining a draw and flush cycle; providing a sensor coupled to the blood access device and configured to generate one or more portions of a signal-time history associated with the draw and flush cycle; and providing a processor for executing computer program code stored in a non-transitory computer-readable medium to cause the processor to perform a series of actions. In an embodiment, the series of actions include operating the flow controller to sequentially draw blood and flush fluid through the blood access device; determining a signal-time history for the draw and flush cycle from the sensor; determining a time duration of a predetermined portion of the signal-time history associated with the draw and flush cycle; determining whether the time duration is greater than a predetermined threshold value; and detecting an occlusion in the blood access device when the time duration of the signal-time history is greater than the predetermined threshold value.

In some embodiments, the sensor is a glucose sensor. In further embodiments, the method includes halting display of information on a display when the occlusion is detected. In still further embodiments, the predetermined threshold value is determined based on a standard signal-time history analyzed when an occlusion is not present.

In a still further aspect, a computer program product for detecting an occlusion in a blood access device is provided. In some embodiments, the computer program product includes a non-transitory computer-readable medium comprising a set of codes for causing a computer to: operate a flow controller to sequentially draw blood and flush fluid through a blood access device, wherein the blood access device is in fluid communication with a calibrant solution source and a circulatory system of a patient; determine a signal-time history for the draw and flush cycle from a sensor, wherein the sensor is coupled to the blood access device; determine a time duration of a predetermined portion of the signal-time history associated with the draw and flush cycle; determine whether the time duration is greater than a predetermined threshold value; and detect an occlusion in the blood access device when the time duration of the signal-time history is greater than the predetermined threshold value.

In some embodiments, the sensor is a glucose sensor. In further embodiments, the computer program product halts display of information on a display when the occlusion is detected. In yet still further embodiments, the predetermined threshold value is determined based on a standard signal-time history analyzed when an occlusion is not present.

The features, functions, and advantages that have been discussed may be achieved independently in various embodiments or may be combined with yet other embodiments, further details of which can be seen with reference to the following description and drawings.

### BRIEF DESCRIPTION OF THE DRAWINGS

Having thus described embodiments of the system in general terms, reference will now be made to the accompanying drawings, where:

FIG. 1 is a perspective view of an analyte sensing system according to one embodiment of the present disclosure;

FIG. 2A is a flow chart showing high-level operation of the analyte sensing system according to one embodiment of the disclosure;

FIG. 2B is a diagram of an analyte signal waveform showing the sensor response of the high-level operation of the analyte sensing system of FIG. 2A according to one embodiment of the disclosure;

FIG. 3 is a flow chart of a method of detecting a clot based on signal-time history diagnostics according to one embodiment of the disclosure;

FIG. 4 is a diagram of a waveform when a clot is not present according to one embodiment of the disclosure;

FIG. 5 is a diagram of a waveform when a clot is present according to one embodiment;

FIG. 6 shows an example of waveform data depicting presence of a clot;

FIG. 7 is a flow chart showing a method of detecting shifts away from a trend in estimated analyte values; and

FIG. 8 is a flow chart showing a method of identifying potential errors in an estimated blood analyte value.

### DETAILED DESCRIPTION OF EMBODIMENTS

Embodiments of the present system now may be described more fully hereinafter with reference to the accompanying drawings, in which some, but not all, embodiments of the system are shown. Indeed, the system may be embodied in many different forms and should not be construed as limited to the embodiments set forth herein; rather, these embodiments are provided so that this disclosure may satisfy applicable legal requirements. Like numbers refer to like elements throughout.

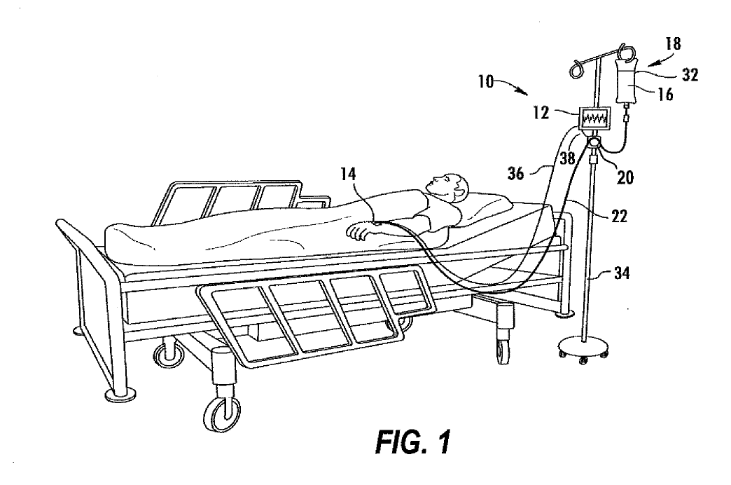

Embodiments of the present disclosure include a blood analyte sensor system 10 that includes a monitor 12, a sensor assembly 14, a calibrant solution source 16 and a flow control system 18, as shown in FIG. 1. The system may also include other sensors, such as pressure sensors, temperature sensors, pH sensors, and the like. Notably, the present disclosure could also be employed with other analyte or blood parameter sensing systems that require drawing of blood or fluid samples from a patient. Blood, as used herein, should be construed broadly to include any body fluid with a tendency to occlude lumens of various body-access devices during sampling. The body access devices include blood access devices such as catheters, tubes, and stents. Generally, the flow control system 18 of one embodiment of the present disclosure is configured to mediate flow of small volumes of the calibrant solution over the sensor assembly 14 and withdraw small volumes of samples of the blood from the patient for testing by the sensor assembly.

The flow control system 18 in another embodiment is able to support the flush and draw pressures and volumes, and the high number of sampling cycles over a long multi-day indwell, needed for continuous analyte (glucose) monitoring, while avoiding the formation of thrombi that occur in conventional catheters by providing a small-diameter, smooth and relatively void free surface defining a lumen extending up to the sensor assembly 14. In still other embodiments of the present disclosure, thrombus formation is inhibited by balancing the structure of various components of the flow control system 18 and operation of the flush and draw cycles by the flow controller 20.

The flow controller 20 in one embodiment of the present disclosure includes some type of hardware, software, firmware or combination thereof that electromechanically controls one or more valves, or other mechanical flow control devices, to selectively allow or stop flow through the monitor line 22. In some embodiments, the mechanical aspect of the flow controller 20 includes a rotary pinch valve through which extends the monitor line 22. This rotary pinch valve pinches the fluid line to stop flow and, by sliding along a short length of the fluid line, can advance or retract the calibrant solution or retract the calibrant solution supply in a column extending down to the end of the catheter. Different numbers of roller heads may be used, such as two or four heads, the latter aiding with higher draw volumes.

In one embodiment, the flow controller 20 employs a combination of the head (primarily, except for the short draw and infusion by pinch point advancement) generated by the elevation of the fluid bag 32 on the pole 34 and the on-off regulation of the flow induced by the head. The flow controller 20, however, could also include a combination of an actual powered pump and its programmable controller, so as to eliminate the need for the pole 34. This pump could be combined with the aforementioned calibrant solution source 16. One advantage, however, of the illustrated embodiment is that the gravity feed of the fluid bag 32 on the pole 34 is well-understood and mediated to control the amount of fluid administered to the patient. Use of active pumps should be controlled in some manner to avoid administration of excess fluid and its side-effects. Regardless, the role of the flow controller 20 can be met flexibly with various combinations of technology and the present disclosure shouldn't necessarily considered limited to any one particular configuration.

The monitor 12 is connected in communication with the sensor assembly 14 through communication lines 36, which may be wires, and to the flow control system 18 through communication lines or wires 38, as shown in FIG. 1. In an embodiment, the monitor and the flow controller are integrated together. The communication lines 36, 38 could also represent wireless data communication such as cellular, RF, infrared or blue-tooth communication. Regardless, the monitor 12 includes some combination of hardware, software and/or firmware configured to record and display data reported by the sensor assembly 14. For example, the monitor may include processing and electronic storage for tracking and reporting blood glucose levels. In addition, the monitor 12 may be configured for automated control of various operations of other aspects of the sensor system 10. For example, the monitor 12 may be configured to operate the flow control system 18 to flush the sensor assembly 14 with calibration solution from calibrant solution source 16 and/or to draw samples of blood for testing by the sensor assembly. Also, the monitor 12 can be configured to calibrate the sensor assembly 14 based on the flush cycle.

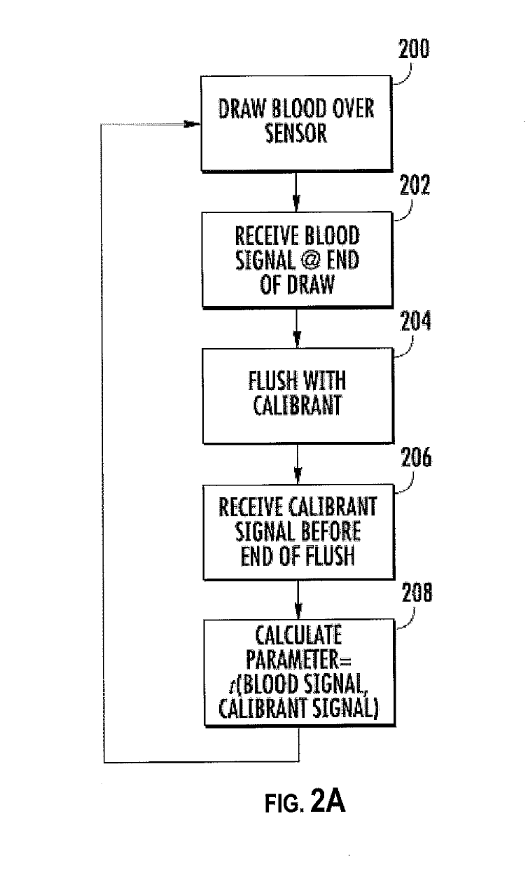

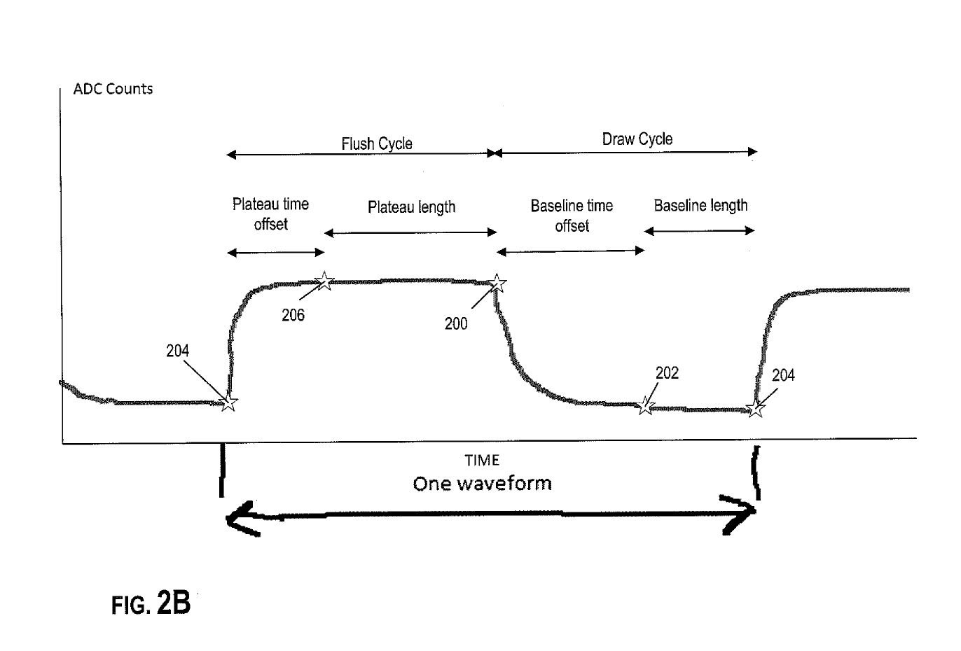

As shown in FIG. 2A, one embodiment the present disclosure may include systems, methods, processes or computer programs for calibrating a blood sensing system and/or operating a blood parameter sensor system. For example, as shown in FIG. 2A, one embodiment of the present disclosure includes drawing blood 200 over a blood parameter sensor, receiving a blood signal 202 near the end of the draw, flushing the sensor with calibrant 204, receiving a calibrant signal 206 before the end of the flush and calculating a blood parameter 208 as a function of both the blood signal and the calibrant signal. It should be understood that the sensor may be flushed with calibrant prior to the system drawing blood over the blood parameter sensor.

FIG. 2B shows a graphical depiction of the magnitude of a current measurement over time as the blood parameter sensor system of FIG. 2A is operated. At point 200, the flow controller draws blood up to the sensor and the draw cycle begins. The draw cycle includes a baseline time offset, which is a predetermined period of time during which the flow controller is drawing blood up to the sensor but the system is not determining an estimated analyte value from the sensor response. The length of the baseline time offset is determined so that calibrant solution may be washed away from the sensor during standard (e.g., non-occluded) operation of the system. At the end of the baseline time offset, the draw cycle includes a baseline length during which a blood signal is received 202. The blood signal is the signal from which the estimated analyte values are determined.

FIG. 2B also includes the flush cycle, comprising a plateau time offset and a plateau length. When the draw cycle concludes, the flow controller causes calibrant solution to flush through the blood access device and wash blood away from the sensor. The plateau time offset, which is a predetermined time period that allows blood to be washed away before calibration measurements begin, starts when the flush begins 204. Again, the duration of the plateau time offset may be determined such that blood is washed away before calibration measurements begin during standard (e.g., non-occluded) operation of the system. After the plateau time offset period ends, the system begins receiving the calibrant signal 206. The calibrant signal is used to determine a sensitivity value for the sensor and determine the estimated analyte value for the waveform.

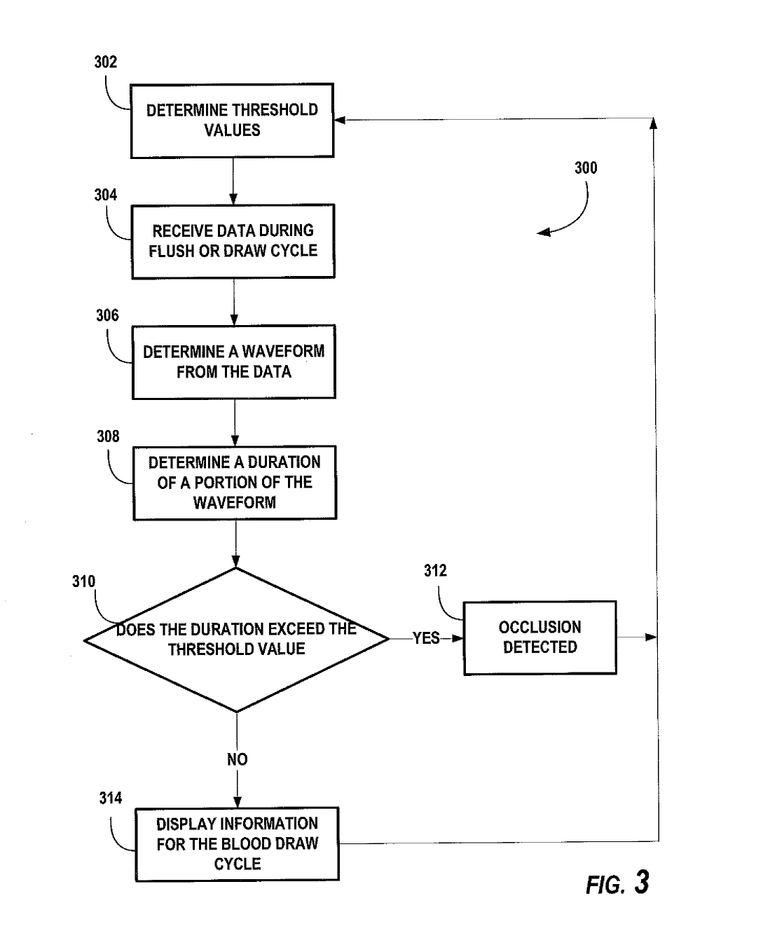

The inventors have observed that continuous analyte monitoring systems employing “one size fits all” flow profiles may be unable to detect and adapt to flow problems. For example, partial or complete clots may occur and occlude the blood access device. The inventors have concluded that an occlusion and hence an inaccurate blood analyte measurement can be detected by evaluating a waveform for diagnostic characteristics, as will be described in FIG. 3. In an embodiment, the clot or occlusion is caused by coagulation on the sensor array. In a further embodiment, the blood access device is occluded. In a still further embodiment, the blood access device may be pressed against the wall of the vein or artery. In some embodiments, the vein or artery collapses and causes an occlusion. Clots and occlusions can be caused by many factors and the system and method disclosed herein can identify them based on diagnostic characteristics of the waveform. The clot or occlusion may be a complete or partial block of flow through the blood access device.

Referring now to FIG. 3, a flow diagram illustrating a method and/or process 300 for detecting an occlusion based on a waveform is provided. Typically, a sensor shows a very quick response to changing analyte concentration during the transition between a flush and a draw. A clot, however, causes the measured analyte value to change more slowly because blood is not being flushed away from the sensor as quickly by calibrant solution and/or blood is not being drawn up to the sensor as quickly. The duration of one or more portions of the waveform can be used to determine whether a clot is present. As discussed in FIG. 2B, a waveform includes multiple portions defined by the flush and draw cycles of the waveform as well as when calibration and blood analyte measurements are made. The portion of the waveform defined by the transition between the flush and the draw cycles typically occurs very quickly but can be affected by clots. If the portion of the waveform corresponding to one of the transition periods occurs over a prolonged duration, the method may determine that a clot is present. For example, the duration to reach a predetermined percentage of the range from the mean calibrant current to the mean baseline current can be used to determine if the transition is occurring under non-occluded conditions.

The process 300 may be implemented as a run-time error checking process. In one embodiment, the process 300 may be implemented as a passive monitoring routine that continuously runs. In such an embodiment, the analyte signal is constantly monitored to detect the occurrence of an occlusion. In a further embodiment, the process 300 may be used to determine when a clot or occlusion has been cleared. In this embodiment, the process 300 may be initiated by the user when a clot has been detected based on the analyte measurement waveform or based on other methods of detecting clots, such as pressure-based systems.

As represented by event 302, in one embodiment, the system receives threshold values for characteristics of a signal-time history (waveform). The thresholds may be received from a user, such as thresholds input by a user via a computing device or graphical user interface. In a further embodiment, the thresholds are determined by the system using waveforms created when an occlusion is known not to be present. For example, the system may determine a standard waveform having a rapid change between draw to flush and an extended plateau and baseline between the transition periods. Using this standard waveform, the system can determine a rate of change of the analyte measurement value and the expected durations for different portions of the waveform. In an embodiment, the system uses these standard durations to determine threshold values. For example, the system may determine that a threshold may be two standard deviations from the median value determined during a standard waveform. This allows for variation in the standard waveform but captures errors that would not occur by chance. Similarly, the thresholds may be determined as a function (e.g., standard deviation, percentage change, scaling, or the like) from the baseline values. In a still further embodiment, the threshold values are determined based on the equipment used, the pressure applied by the flow controller, the diameter of the blood access device, and/or the blood pressure of the patient.

The thresholds may include the length of time to complete a predetermined amount of the transition (e.g., median or 90% of the transition period) between the calibration and blood measurement periods. As discussed, typically the transition periods of the flow cycle occur very quickly. A clot, however, will cause extended mixing of blood and calibration solution during at least one of the transition periods and cause an increasingly slow sensor time response.

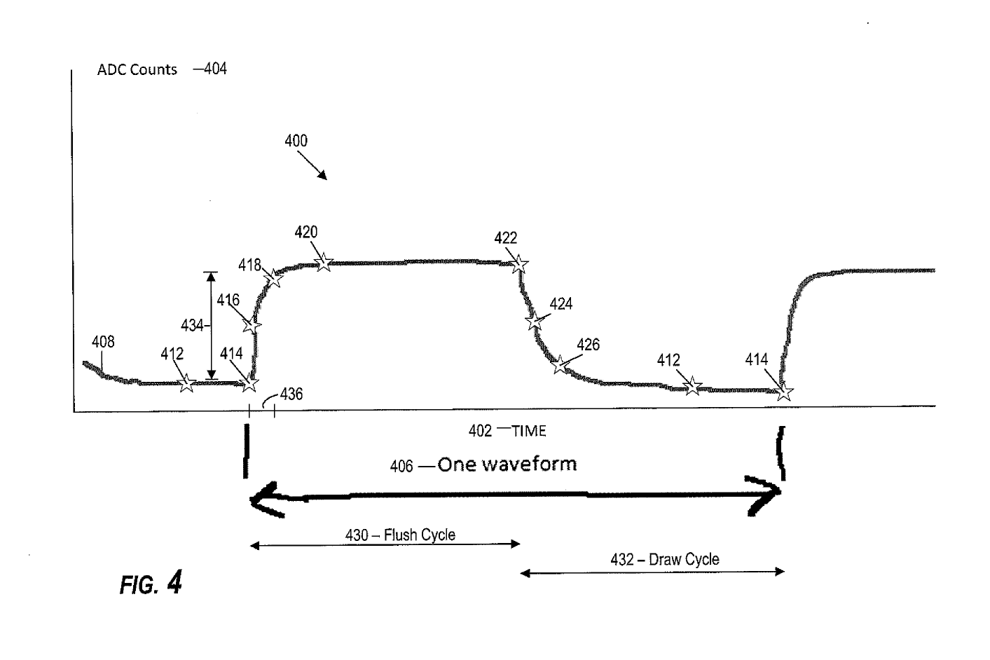

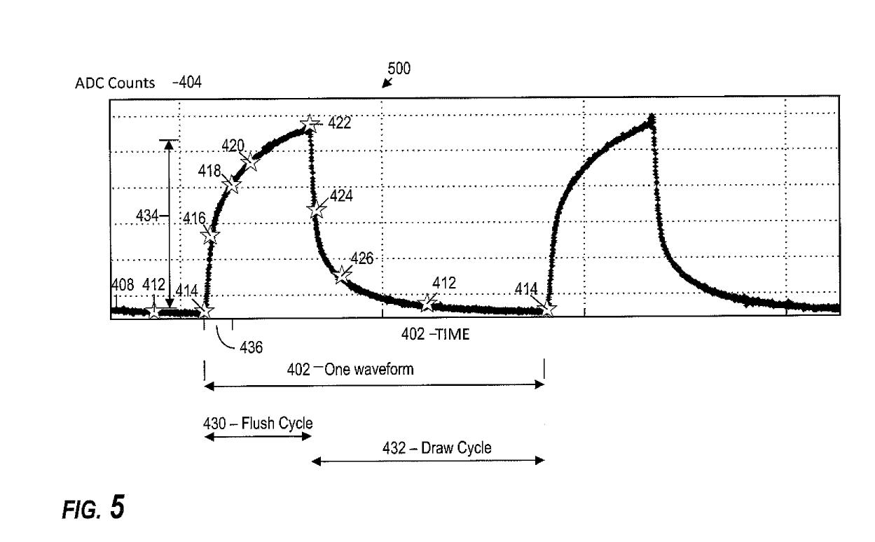

Turning briefly to FIGS. 4 and 5, a comparison of signal-time history waveforms derived from an occlusion-free cycle 400 and an occluded cycle 500 is provided. In FIGS. 4 and 5, the waveforms 406 are determined from signals identified by a sensor. In an embodiment, the signals correspond to glucose concentration but it should be understood that the method may be used with other types of analyte sensors. The waveforms 406 provide a chart of analog-to-digital converter count 404 over time 402. The analog-to-digital converter (ADC) counts 404 are measurements of values received from the analyte sensor during the flush and draw cycles. The ADC count 404 converts an input current to a digital number proportional to the magnitude of the current and can be used to determine changes in analyte concentration in a fluid by evaluating changes in the magnitude of the current over time.

The waveform 406 includes a flush cycle 430 and a draw cycle 432. During the flush cycle 430, the flow controller advances calibration solution through the blood access device to wash blood away from the sensor and bathe the sensor in calibration solution, allowing a calibration period to occur and sensitivity measurements to be made. During the draw cycle 432, the flow controller draws blood from the circulatory system of the patient through the blood access device and into contact with the sensor so that blood analyte concentrations may be measured.

The flush cycle 430 starts at a point where blood clear begins 414 and ends at the end of a calibration period 422. The flush cycle includes a plateau time offset starting from the point where the blood clear begins 414 and extending to the end of the duration of the plateau time offset 420. In an embodiment, the duration of the plateau time offset is provided by the user or the system. The flush cycle also includes a plateau length during which the calibration occurs. When an occlusion is not present and the calibrant solution is a constant concentration, the plateau length has a relatively constant current (via ADC count). The plateau length extends from the time the plateau time offset ends 420 to the end of the calibration 422. When an occlusion is present, the plateau may be sloped.

The draw cycle 432 begins at the end of the calibration period 422 and includes a baseline time offset and a baseline time length during which blood measurement occurs. The baseline time offset starts from the end of the calibration period 422 through the duration of the baseline time offset 412. The baseline time length is a predetermined period of time during which the sensor is recording current data so that analyte values may be determined for the blood, and extends from the end of the baseline time offset 412 to the end of the blood draw and beginning of the blood clear 414.

The duration of the plateau and baseline time offsets and lengths of time for the plateau and the baseline may be input by the user, based on blood characteristics of the patient, dependent upon sensor run-in, or determined based on other variables related to the sensor system.

In some embodiments, the system receives signals from the analyte sensor during each of the periods disclosed herein, i.e., the plateau time offset, the plateau length, the baseline time offset, and the baseline length. The individual signals have a magnitude corresponding to the current measured by the sensor at the time the signal was detected. The system is able to determine the magnitude of each signal, the time when each signal occurred, the number of signals in any given period, and transformations of the signals for the periods. For example, in some embodiments the system determines the mean current magnitude during the plateau length, corresponding to the calibration current, and the mean current magnitude during the baseline length, corresponding to the blood current. The system is able to determine the absolute difference between the mean calibration current and the mean blood current to determine the range in current magnitude values between the calibration solution and the blood.

When an occlusion is not present, the waveform quickly reaches a plateau and stays at the plateau for the duration of the flush cycle, as shown in FIG. 4. As can be seen based on a comparison of FIG. 4 to FIG. 5, when a clot occurs the waveform 406 takes on a characteristic “shark fin” shape. When an occlusion is present the waveform does not reach the plateau quickly but instead slowly increases or decreases because of the reduced flow rate and continued mixing of blood and calibrant solution. It should be understood that the waveform may be inverted if the analyte concentration of the calibration solution is less than the analyte concentration of the blood.

Turning back to FIG. 3, at event 304, the system receives data during a flush and/or draw cycle. In an exemplary embodiment, the system causes the flow controller to sequentially flush fluid through the blood access device and then draw blood up to the sensor via the blood access device. The system continuously monitors the sensor output during the flush and draw cycles.

At event 306, the system determines a waveform produced by the analyte signal during the draw and/or flush cycle. In one embodiment, a waveform corresponding to a glucose concentration is determined continuously during both the blood draw phase and the calibrant solution flush phase. In some embodiments, the system receives data from two or more sensors. The analyte sensor output data may be one or more analyte signals that produce an analyte concentration curve. The analyte concentration curve may be produced by linear regression, smoothing algorithms, splicing algorithms, or any other method of creating a curve from a plurality of points. For example, the analyte concentration curve may be determined by connecting adjacent pressure data points with a straight line. Data related to other blood sensing parameters may also be extracted during event 306. For example, pressure measurements from a pressure sensor may also be received.

It should be understood that a waveform is a convenient graphical depiction of a set of data that can be used to evaluate whether a clot is present. The waveform represents a signal-time history of signals received by the system during the flush and draw cycles. While the disclosure presented herein discusses analyzing a waveform, it should be understood that the system may analyze the data underlying the waveform to determine whether a clot is present.

At event 308, the system determines a duration of a portion of the waveform. In an embodiment, the duration of predetermined portions of the waveform are diagnostic of clots. For example, the duration to go from the beginning of the blood clear 414 to the median of the transition period is very short when no clot is present but has an extended duration when a clot is present. In an embodiment, multiple portions of the waveform are evaluated to determine whether a clot is present. For example, the product of durations in both transitional periods of a waveform may be determined. In an exemplary embodiment, the system determines the length of time for the system to reach the median of the transition time and/or the point where 90% of the transition is completed.

In some embodiments, the characteristic is scaled based on an initial measurement. In a still further embodiment, the characteristic is a function of multiple time measurements. For example, the characteristic may be the product of the length of time to reach 90% of the transition from the draw to the flush and the length of time to reach 90% of the transition from the flush to the draw. The location of the occlusion and the stage of the flush and draw cycle at which the occlusion forms may cause one of the flush to draw or draw to flush transitional periods to be extended while not extending the other transitional period.

The time to reach 90% and the time to reach the median of the transitional periods will be used as example durations for use by the system. In an embodiment, the time to reach 90% of the transition between the flush and draw is termed the T90 value. In an embodiment, both the transition period from the flush to the draw and the transition period from the draw to the flush have a T90 value, termed Flush T90 and Draw T90 respectively. The flush T90 is determined by finding the point at which the blood clear begins 414 and finding the point at which the calibration period ends 422. Both points are known by the system based on the flow controller history. The duration of time between these two points is the duration of the flush cycle. Values for the current can be determined for the flush duration, including individual current values (e.g., magnitude), mean values, measurements of variation (e.g., coefficient of variation), and the like.

In an embodiment, current values are also determined for the calibration period based on the time the plateau time offset ends 420 and the time the calibration period ends 422. In a still further embodiment, current values are determined for the blood measurement period based on the time the baseline time offset ends 412 and the time the blood clear begins 414. In one embodiment, the difference between the mean plateau current and the mean baseline current is determined to determine the range 434 in current magnitude from the calibration fluid current to the blood current. The range 434 will be used to determine the sensor response time by determining how quickly the sensor response changes relative to the range.

In an embodiment, the difference between current over the entire duration of the flush cycle and the current during the calibration period is determined. For example, current values that correspond to the calibration period can be excluded from the flush cycle to produce a set of current values that only corresponds to the period of time between the start of blood clearing 414 and the start of the calibration period 420. In an embodiment, the absolute value of the difference of the set of current values between the flush cycle current and the calibration period current is the set of current values during the transitional period.

In a further embodiment, the system determines which current values are below a predetermined value compared to the range 434. For example, the system may determine which current values from the transitional period are less than or equal to 90% of the magnitude range from the mean blood current to the mean calibration current. The system then determines the duration that the determined current values represent. The system determines the earliest time point for a current value that is less than or equal to 90% of the total range and the latest time point for a current value that is less than or equal to 90% of the total range. The duration from the earliest to the latest time point is a duration of a portion of the waveform that will later be compared to thresholds to determine if a clot is present.

For exemplary purposes, the range 434 may be represented by a value of 100 ADC. This is the difference in ADC value between the mean calibration and the mean blood values. Ninety percent of the range is 90 ADC. The system identifies which individual current magnitude values during the transition period are less than or equal to 90 ADC above the baseline. The system then determines the duration over which these identified individual current values were received. This duration is the length of time to reach a predetermined amount, e.g., 90%, of the difference between the baseline and the calibration. Without a clot, this occurs very quickly. With a clot, this duration is extended due to mixing of blood and calibration solution.

The determination is also illustrated in FIGS. 4 and 5. The range 434 between the mean current during the calibration period (from 420 to 422) and the mean current during the blood measurement period (from 412 to 414) is determined based on the flow controller start and stop time as well as the plateau and baseline time offsets and lengths. The system determines how long it takes for the current to reach predetermined levels in the range, e.g., 50% of the range or 90% of the range. The system may determine the current values between 414 and 422. The system excludes current values determined between 420 and 422 to identify current values during the transitional period. The system may identify which of the current values during the transitional period are less than or equal to 90% of the range in current between the calibration period and the blood measurement period. In an embodiment, point 418 identifies the last current value that is determined to be less than or equal to 90% of the range. The system determines the duration 436 to go from the start of the blood clear 414, where the first current value which is less than or equal to 90% of the range is located, and point 418. This duration can be compared to a threshold value to determine whether a clot is present.

In FIG. 4, the duration 436 to go from the start of the blood clear to 90% of the transition period is short because the waveform quickly reaches the plateau as blood is washed away easily. In FIG. 5, however, the duration 436 is longer because of the gradual increase during the transitional period. Similarly, point 416 may be the last current measurement which is below 50% of the range from the mean calibration current to the mean blood measurement current. It should be understood that both transition periods in a waveform may be analyzed in this manner. FIGS. 4 and 5 include points 424 and 426, which represent the last current value which is 50% and 90% of the range from the mean calibration current to the mean blood measurement current. Again, the duration to reach these points from the end of the calibration period 422 can be determined and compared to a threshold. If no clot is present, the waveform will display a rapid decrease in ADC count and the duration to reach 50% or 90% of the range will be short. If a clot is present, however, the duration is extended due to mixing.

At event 310, the duration of the portion of the waveform, determined at event 308, is compared to the threshold value, determined at event 302. In one embodiment, the threshold value may be a predetermined value, for example values based on flow controller, such that when the duration exceeds this value an occlusion is determined to be present. In an embodiment, the threshold is based on the diameter of a lumen in the blood access device and/or the flow rate through the blood access device. In a further embodiment, the threshold is based on the pressure applied by the flow controller. In a still further embodiment, the threshold may be determined at least in part by an internal pressure for the blood access device, the patient, a body lumen in the patient, or other component of the dynamic fluid system comprising the flow controller, the blood access device, and the patient. It should be understood that the threshold may be determined by a user, by the manufacturer of a component of the system, or by the system itself, and that other methods of determining the threshold are possible.

If the duration of the portion of the waveform is determined to be greater than the threshold value then an occlusion is detected at event 312. The presentation of information, such as temperature, volume, or pH, may be suspended when the measurement of pressure exceeds the threshold value. The system may also take remedial measures to clear the occlusion.

At event 314, if the duration is determined to be less than the threshold value then an occlusion in not indicated based on the waveform and information determined during the blood measurement period may be presented on the monitor. This event may also be used to determine when an occlusion has been cleared. For example, the system may be activated when an occlusion is detected and used to evaluate when the occlusion is cleared.

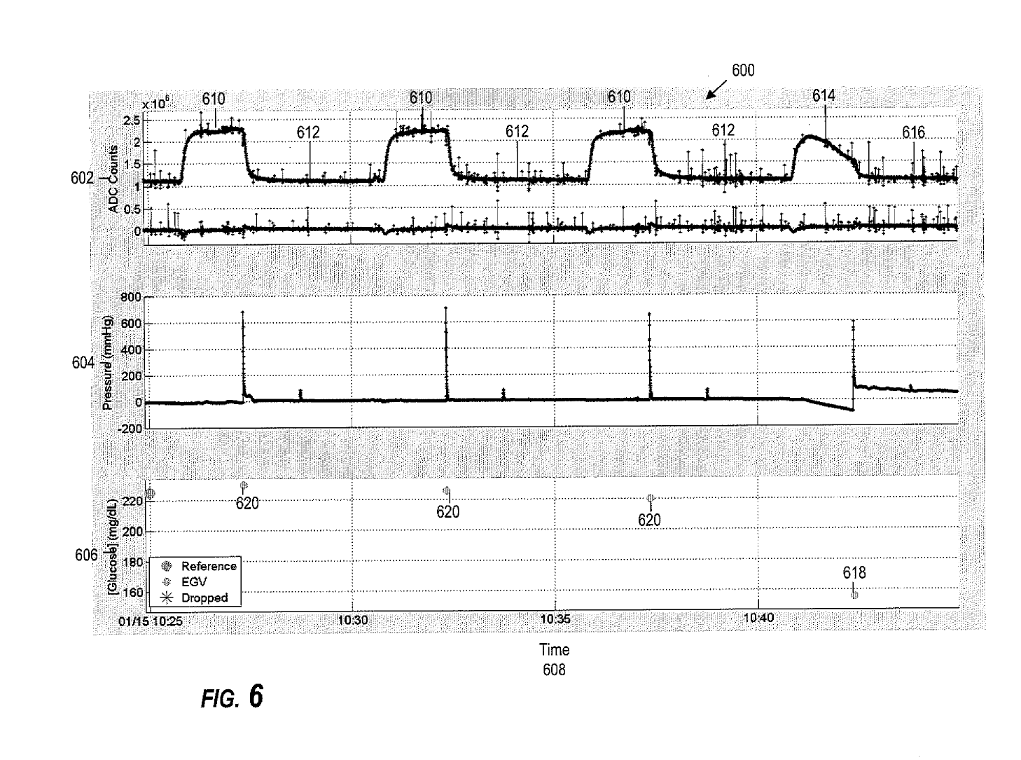

Turning now to FIG. 6, an exemplary graph 600 of analyte sensor output 602, pressure 604 signals, and estimated glucose values 606 over time 608 during sequential flush and draw cycles is provided. The analog-to-digital converter (ADC) counts from the analyte sensor output 602 are measurements of the values received from the glucose analyte sensor during the flush and draw cycles. Pressure signals 604 are determined and glucose values 606 are estimated at the same time as the ADC counts. The pressure curve is defined by a plurality of pressure signals 604. In an embodiment, the estimated glucose values 606 are determined based on sensitivity values determined from the calibration periods.

In FIG. 6, three plateaus 610 from calibration periods correspond to three flush cycles that do not have an occlusion. The waveform pattern for each of these plateaus 610 and corresponding baseline blood measurement periods 612 do not have diagnostic characteristics indicating a clot, e.g., a “shark fin” shape. Instead, the waveforms during these cycles show fast transition periods between the draw and the flush cycles.

In contrast, one plateau 614 shows the characteristic “shark fin” shape indicating a possible occlusion. The system is able to determine the mean current during the calibration period and the mean current during the baseline blood measurement period 616. The system identifies the current values during the transition periods on both sides of the plateau 614. The system then determines the current values that are less than or equal to a predetermined amount of the range, e.g., 50% or 90%. The duration to go from the beginning of the blood clear to the predetermined value or the end of the calibration period to the predetermined value is determined and compared to threshold values. In this example, plateau 614 includes a draw cycle having a slow sensor response, likely due to an occlusion. The occlusion is indicated by a longer duration for the decline during the transition period. Correspondingly, the estimated glucose value 618 for the waveform is clearly different compared to the estimated glucose values 620 determined during the plateaus 610 non-occluded cycles.

In an embodiment, when the waveform characteristic is greater than a predetermined threshold, presentation of information on a monitor is suspended. When the waveform characteristic is less than the predetermined threshold, information may be presented on the monitor again.

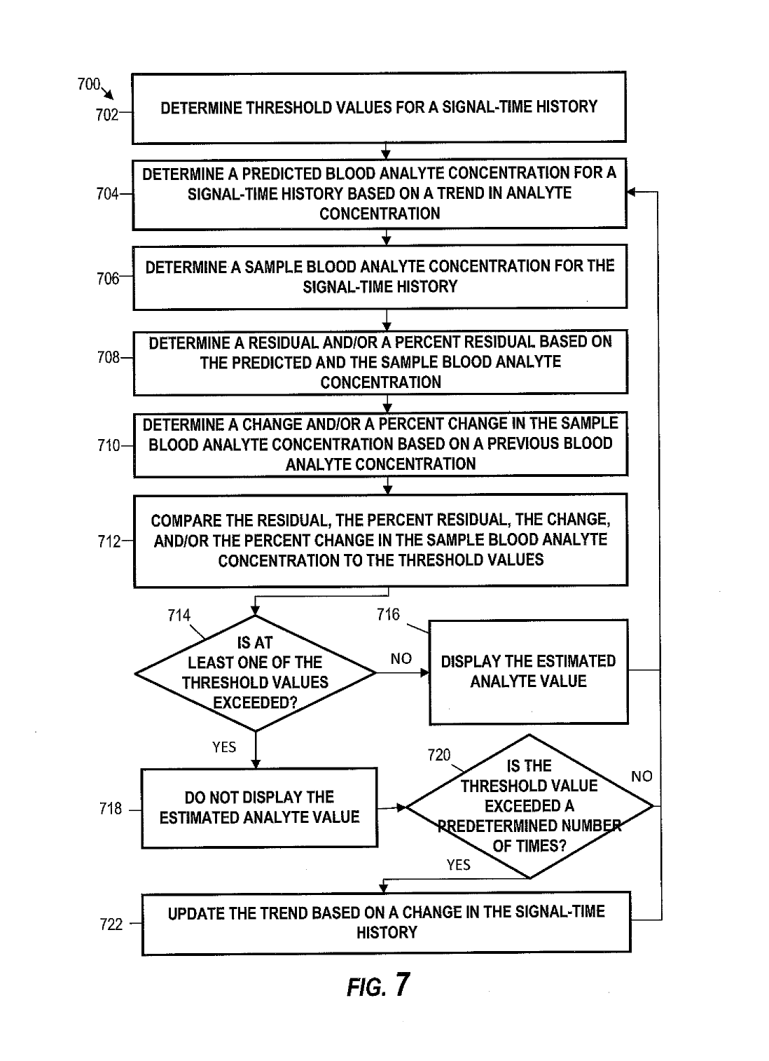

More generally, the signal-time history may be used to evaluate the accuracy and reliability of estimated analyte values, or to indicate potential problems in the sensor response. For example, sensor signals that are outside of an expected range may indicate an error with the sensor, the calibrant, or the blood access device. In an embodiment disclosed in FIG. 7, a method of detecting shifts away from a trend in estimated analyte values is provided. In the method 700, a trend in the signal-time history is determined to produce a predicted estimated analyte value. A residual of the difference between the estimated and measured analyte value can be compared to a threshold residual to determine whether the measured analyte value is very different from the predicted value, possibly indicating an error in the measurement apparatus. Other characteristics of the signal-time history may also be evaluated, such as a change in the analyte value compared to a recent value, as further indications of potential issues in the measurement.

In block 702, the method determines threshold values for a signal-time history. The threshold values may be input by a user of the method. In some embodiments, the threshold values are determined by the system based on an evaluation of multiple signal-time histories. In some embodiments, a system implementing the method has pre-set threshold values. In an embodiment, the thresholds are dependent at least in part on the patient. For example, a patient may have stable blood glucose levels and therefore have a reduced threshold compared a patient with unstable blood glucose levels. In further embodiments, the threshold values are determined based on a combination of user input-values, machine-dependent characteristics, and patient-dependent characteristics. The threshold values may be changed by the user, such as after the sensor is run-in or if the threshold values are triggering frequently compared to other measurements of error in the measurement cycle. In an embodiment, the threshold values for the signal-time history include a residual threshold, a percent residual threshold, a change in analyte concentration threshold, a percent change in analyte concentration threshold, and/or an extreme residual limit threshold. It should be understood that other types of threshold values may be determined, such as other types of measurements of difference between a predicted value and a measured value. The threshold value may be an absolute value, such as a value for glucose concentration measured in mg/dL, or it may be a relative value, such as a 10%, 20%, 30% or other percentage increase compared to a standard, known, or trusted value.

In block 704, the method determines a predicted blood analyte concentration for a signal-time history based on a trend in analyte concentration. In some embodiments, the trend in the analyte concentration is determined using a second-order polynomial fit. Other methods of fitting a line, whether linear or non-linear, may be used to determine a trend. In an embodiment, the trend is based on data from a previous blood analyte measurement period. For example a previous blood analyte measurement period where all data were within acceptable ranges based on comparison to threshold values may be used to determine a trend for a present blood analyte period. When the trend is determined, a predicted value based on the trend can be determined. For example, the trend can be extrapolated forward to the present blood analyte measurement period and a predicted blood analyte concentration can be determined based on the previous blood analyte concentrations. Typically, blood analyte concentrations change slowly such that a trend, e.g., increasing, decreasing, remaining relatively stable, is likely to continue at a relatively constant rate.

In block 706, the method determines a sample blood analyte concentration for the signal-time history. As discussed previously, the sample blood analyte concentration is determined based on signals received from the analyte sensor during a flush and draw cycle. For example, the sample blood analyte concentration may be determined by dividing the current determined during the blood draw by a sensitivity value determined during calibration cycle. In some embodiments, the sample blood analyte concentration is modified by a constant, such as a value determined from a non-analyte sensor or a bias value.

Turning now to block 708, the method determines a residual and/or a percent residual based on the predicted and sample blood analyte concentration. The residual is an estimate of the error based on the comparison of the predicted and sample blood analyte concentrations. In an embodiment, the residual is determined by taking the absolute value of the sample concentration minus the predicted concentration. The percent residual is the absolute value of the residual divided by the sample blood analyte concentration. The residual and the percent residual are measurements of error based on the comparison of the predicted value and the sample value and can be compared to threshold values to determine whether the blood sample resulted in a value that is greater than expected.

In block 710, the method determines a change and/or a percent change in the sample blood analyte concentration based on a previous blood analyte concentration. Similar to a comparison based on a predicted value from a trend, a comparison to a previous blood analyte concentration can be used to determine if the present blood analyte concentration has diverged more than expected. In an embodiment, the previous blood analyte concentration is a blood analyte concentration from a flush and draw cycle that is within expected parameters. In an embodiment, the previous blood analyte concentration is the most recent displayed analyte value, wherein analyte values are displayed if they are considered accurate based on measurements of error during the flush and draw cycle. In some embodiments, the measurement of change is the absolute value of the difference between the present blood analyte concentration and the most recent displayed value. In an embodiment, the percent change in the sample blood analyte concentration is the change in the blood analyte concentration divided by the most recently displayed value.

In block 712, the method compares the residual, the percent residual, the change, and/or the percent change in the sample blood analyte concentration to the threshold values. The comparison may be a direct comparison of the value determined for the cycle compared to the values determined in block 702. In an embodiment, the user is queried regarding whether the user desires to update the thresholds.

In decision block 714, the method determines whether at least one of the threshold values is exceeded. For example, the method determines if at least one, at least two, at least three, or all of the characteristics of the present blood measurement exceed the threshold values. If none of the threshold values are exceed, as shown in block 716, then the method displays the estimated analyte value and continues in the flush and draw cycle.

If, however, at least one of the threshold values is exceeded or if all of the threshold values are exceeded, as shown in block 718, then the estimated analyte value for the blood draw cycle is not displayed. By determining that the threshold values are exceeded, the method determines that the trend indicates a potential issue in the blood analyte value determined during the present cycle. The method may also cause an alarm, such as an audible or visual alarm to be presented to a user.

In block 720, the method determines whether the threshold value is exceeded a predetermined number of times. For example, the method may determine whether a threshold value is exceeded in four consecutive cycles. In some embodiments, the method determines whether the same threshold is exceeded in consecutive cycles. Continuously exceeding a threshold may indicate an underlying change in the analyte values from previous analyte values instead of an error in measurement. For example, a medicine may be administered to a patient that changes analyte concentration in the blood. If the threshold and comparisons are based on blood measurements taken before administration of the medicine, the thresholds may be exceeded because the blood analyte concentration is changing instead of an error, such as an occlusion, in the blood access device. If the threshold value is not exceeded a predetermined number of times, the method continues to evaluate the flush and draw cycles for potential issues in the blood analyte concentration.

If, however, the threshold value is exceed a predetermined number of times, then the method updates the trend based on a change in the signal-time history, as shown in block 722. For example, the threshold values and/or the predicted value may be changed based on more recent data received from the blood analyte sensor. In this manner, underlying changes in blood chemistry can be reflected in the method. Thus, a responsive system and method is provided that evaluated trends in blood analyte values and determines whether sample blood analyte concentrations exceed threshold values.

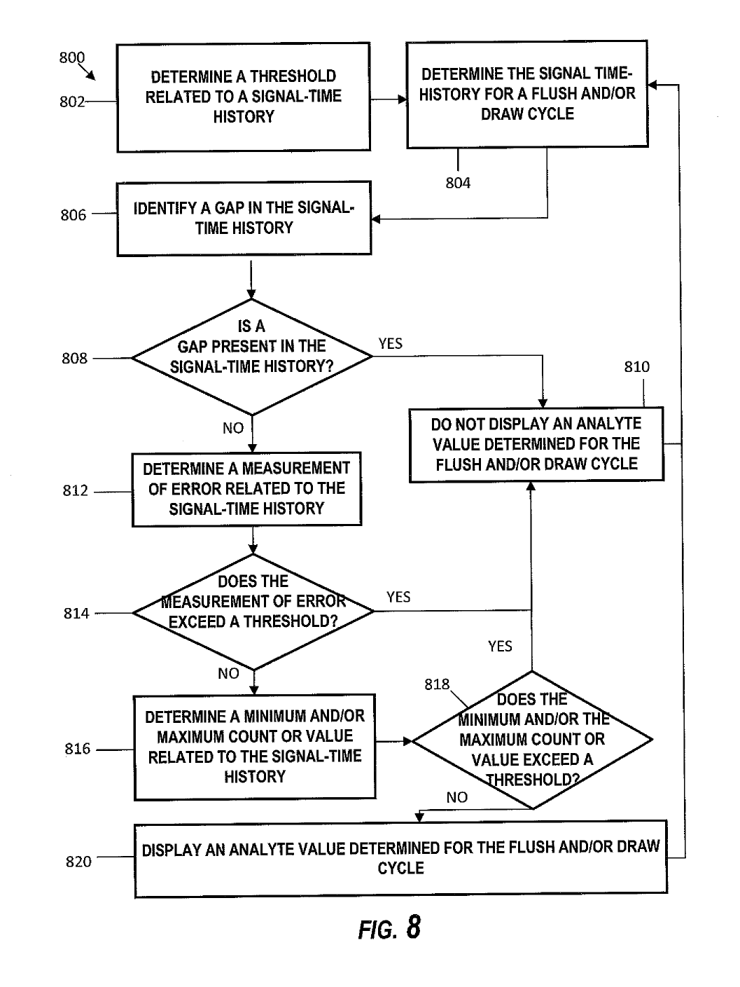

In a further aspect shown in FIG. 8, different types of thresholds may be evaluated to determine whether a potential error in the estimated blood analyte value has occurred. For example, measurements of error in the counts or values may be evaluated to determine if excessive noise is present in the data received during the flush or draw cycle. Minimum and maximum values or counts determined during the flush or draw cycles may also be evaluated to determine if a potential error has occurred. In some embodiments, the characteristics of the signal-time history are compared to threshold values. In an embodiment, gaps in data received during the flush or draw cycle are identified as indicating a potential error in the estimated blood analyte value. The thresholds and data used to identify potential errors in an estimated blood analyte value may be any of the types of data received during the flush and/or draw cycle. For example, in an exemplary embodiment, the data includes analyte sensor data (e.g., blood glucose) as well as pressure data. The data may be raw data, filtered data, or modified data. For example, both ADC counts and estimated glucose values may be used to identify potential errors.

In FIG. 8, a method of identifying potential errors in an estimated blood analyte value is provided. The method includes determining a threshold related to a signal-time history, determining the signal-time history for a flush and/or draw cycle, and performing a series of checks to evaluate the signal time history. The series of checks may include a check for gaps in the data received in the signal-time history, too large or too small measurements of error for data received during the signal-time history, and/or minimum and maximum values related to the signal time history. In an embodiment, the series of checks may determine whether the signal-time history has a characteristic that is greater than a threshold.

In block 802, the method determines at least one threshold related to a signal-time history. The threshold values may be input by a user. In some embodiments, the threshold values are determined by the system based on an evaluation of multiple signal-time histories. In some embodiments, a system implementing the method has pre-set threshold values. In an embodiment, the thresholds are dependent at least in part on the patient. For example, a patient may have stable blood glucose levels and therefore have a reduced threshold compared a patient with unstable blood glucose levels. In further embodiments, the threshold values are determined based on a combination of user input-values, machine-dependent characteristics, and patient-dependent characteristics. The threshold values may be changed by the user, such as after the sensor is run-in or if the threshold values are triggering frequently compared to other measurements of error in the measurement cycle. In an embodiment, the threshold values for the signal-time history include a length of a maximum length of a pressure data gap, a maximum length of an enzyme data gap, a maximum length of a no enzyme data gap, a minimum enzyme signal, a maximum enzyme signal, a maximum no enzyme signal, a maximum measurement of error associated with the analyte concentration or enzyme signal, a minimum estimated analyte value, and/or a maximum estimated analyte value. It should be understood that other types of threshold values may be determined, such as other types of gaps, measurements of variation, or ranges. The threshold value may be an absolute value, such as a value for glucose concentration measured in mg/dL, or it may be a relative value, such as a 10%, 20%, 30% or other percentage increase compared to a standard, known, or trusted value.

In block 804, the method determines the signal-time history for a flush and/or draw cycle. The signal-time history is a record of the signals received from one or more sensors over time during the flush and/or draw cycle. As discussed, the signals may be related to an analyte concentration, a pressure value, or other measurement associated with the flush and/or draw (e.g., temperature, pH, or the like).

In block 806, the method identifies a gap in the signal time history. A gap is a portion of the signal-time history where no data is received for a particular sensor. In an embodiment, the sensor-time history is evaluated to identify gaps in pressure data, enzyme data, and/or no enzyme data.

In decision block 808, the method determines whether a gap is present in the signal-time history. In an embodiment, a gap is present if one or more data points are not present in the data. For example, the analyte sensor may be configured to receive a count on a predetermined schedule. If one of the counts is not received, the method may determine that a gap is present. In another embodiment, a gap is identified based on a length of time. For example, a gap in pressure data may be identified as having a duration. The duration of the pressure data gap is compared to a threshold value to determine whether the duration exceeds the threshold value. The method may evaluate the signal-time history based on absolute gaps or gap length. In a further embodiment, the method may evaluate the signal-time history for relative gaps, or gaps in sensor measurements relative to a previous frequency.

If a gap is detected in block 808, then the method causes the analyte value determined for the flush and/or draw cycle to not be displayed, as shown in block 810. In a further embodiment, an audible or visual alarm, or record, is made of the event. The system may continue to track signal-time histories, as shown in block 804, while preventing display of analyte values calculated during a cycle that fails a check of the method.

In block 812, the method determines a measurement of error related to the signal-time history. The measurement of error may be a coefficient of variation for signals received from a sensor during a flush and/or draw cycle. For example, the current measurements received during a blood measurement cycle comprise individual values in a population. The population has a mean, a standard deviation, and a coefficient of variation. The coefficient of variation is determined by dividing the standard deviation by the mean of the blood current, and is a measurement of noise in the signal. Other measurements of error may be used, such as standard deviation, standard error, or others.

In block 814, the method determines whether the measurement of error exceeds a threshold. For example, the coefficient of variation for the analyte measurement values can be compared to a threshold coefficient of variation to determine whether too much noise is present in the signal. If the coefficient of variation exceeds the threshold, the sample may have been contaminated, such as via an occlusion or defect in the flush or draw. When the measurement of error exceeds the threshold, the analyte value determined for the flush and/or draw cycle is not displayed to the user, as shown in block 810.

In block 816, the method determines a minimum or maximum count or value related to the signal-time history. For example, the method may identify a maximum enzyme signal, a maximum no enzyme signal, a maximum estimated glucose value, a minimum enzyme signal, and a minimum estimated glucose value. The maximum and minimum counts and/or values may be for a predetermined portion of the signal-time history. For example, the maximum may be a maximum value for the blood draw period of the signal-time history or the calibration period of the signal-time history.

In decision block 818, the method determines whether the minimum or maximum counts or values exceed the threshold value. In an embodiment, the system directly compares the measured value to determine whether a threshold is exceeded. In some embodiments, the method may take the absolute value of the measured value to determine whether a threshold is exceeded. If the threshold is exceeded, then the method does not display an analyte value determined for the flush and/or draw cycle, as disclosed in block 810.

If, however, the minimum and maximum count or value is not exceeded, then the method displays an analyte value determined for the flush and/or draw cycle, as shown in block 820. In a further embodiment, a record of the analyte value and/or a history of the checks is maintained by the method. For example, a history of the measurements of error for each flush and/or draw cycle may be preserved.

It should be understood that the checks performed by the method 800 may be performed in any order. For example, the method may first identify minimum and maximum values and only later check for gaps in the data.

Although many embodiments of the present system have just been described above, the present system may be embodied in many different forms and should not be construed as limited to the embodiments set forth herein; rather, these embodiments are provided so that this disclosure will satisfy applicable legal requirements. Also, it will be understood that, where possible, any of the advantages, features, functions, devices, and/or operational aspects of any of the embodiments of the present system described and/or contemplated herein may be included in any of the other embodiments of the present system described and/or contemplated herein, and/or vice versa. In addition, where possible, any terms expressed in the singular form herein are meant to also include the plural form and/or vice versa, unless explicitly stated otherwise. Accordingly, the terms “a” and/or “an” shall mean “one or more,” even though the phrase “one or more” is also used herein. Like numbers refer to like elements throughout.

As will be appreciated by one of ordinary skill in the art in view of this disclosure, the present system may include and/or be embodied as an apparatus (including, for example, a system, machine, device, computer program product, and/or the like), as a method (including, for example, a method, computer-implemented process, and/or the like), or as any combination of the foregoing. Accordingly, embodiments of the present system may take the form of an entirely method embodiment, an entirely software embodiment (including firmware, resident software, micro-code, stored procedures in a database, etc.), an entirely hardware embodiment, or an embodiment combining method, software, and hardware aspects that may generally be referred to herein as a “system.” Furthermore, embodiments of the present system may take the form of a computer program product that includes a computer-readable storage medium having one or more computer-executable program code portions stored therein. As used herein, a processor, which may include one or more processors, may be “configured to” perform a certain function in a variety of ways, including, for example, by having one or more general-purpose circuits perform the function by executing one or more computer-executable program code portions embodied in a computer-readable medium, and/or by having one or more application-specific circuits perform the function.

It will be understood that any suitable computer-readable medium may be utilized. The computer-readable medium may include, but is not limited to, a non-transitory computer-readable medium, such as a tangible electronic, magnetic, optical, electromagnetic, infrared, and/or semiconductor system, device, and/or other apparatus. For example, in some embodiments, the non-transitory computer-readable medium includes a tangible medium such as a portable computer diskette, a hard disk, a random access memory (RAM), a read-only memory (ROM), an erasable programmable read-only memory (EPROM or Flash memory), a compact disc read-only memory (CD-ROM), and/or some other tangible optical and/or magnetic storage device. In other embodiments of the present system, however, the computer-readable medium may be transitory, such as, for example, a propagation signal including computer-executable program code portions embodied therein.

One or more computer-executable program code portions for carrying out operations of the present system may include object-oriented, scripted, and/or unscripted programming languages, such as, for example, Java, Perl Smalltalk, C++, SAS, SQL, Python, Objective C, JavaScript, and/or the like. In some embodiments, the one or more computer-executable program code portions for carrying out operations of embodiments of the present system are written in conventional procedural programming languages, such as the “C” programming languages and/or similar programming languages. The computer program code may alternatively or additionally be written in one or more multi-paradigm programming languages.

Some embodiments of the present system are described herein with reference to flowchart illustrations and/or block diagrams of apparatus and/or methods. It will be understood that each block included in the flowchart illustrations and/or block diagrams, and/or combinations of blocks included in the flowchart illustrations and/or block diagrams, may be implemented by one or more computer-executable program code portions. These one or more computer-executable program code portions may be provided to a processor of a general purpose computer, special purpose computer, and/or some other programmable data processing apparatus in order to produce a particular machine, such that the one or more computer-executable program code portions, which execute via the processor of the computer and/or other programmable data processing apparatus, create mechanisms for implementing the steps and/or functions represented by the flowchart(s) and/or block diagram block(s).

The one or more computer-executable program code portions may be stored in a transitory and/or non-transitory computer-readable medium (e.g., a memory, etc.) that can direct, instruct, and/or cause a computer and/or other programmable data processing apparatus to function in a particular manner, such that the computer-executable program code portions stored in the computer-readable medium produce an article of manufacture including instruction mechanisms which implement the steps and/or functions specified in the flowchart(s) and/or block diagram block(s).

The one or more computer-executable program code portions may also be loaded onto a computer and/or other programmable data processing apparatus to cause a series of operational steps to be performed on the computer and/or other programmable apparatus. In some embodiments, this produces a computer-implemented process such that the one or more computer-executable program code portions which execute on the computer and/or other programmable apparatus provide operational steps to implement the steps specified in the flowchart(s) and/or the functions specified in the block diagram block(s). Alternatively, computer-implemented steps may be combined with, and/or replaced with, operator- and/or human-implemented steps in order to carry out an embodiment of the present system.

While certain exemplary embodiments have been described and shown in the accompanying drawings, it is to be understood that such embodiments are merely illustrative of and not restrictive on the broad system, and that this system not be limited to the specific constructions and arrangements shown and described, since various other changes, combinations, omissions, modifications and substitutions, in addition to those set forth in the above paragraphs, are possible. Those skilled in the art will appreciate that various adaptations, modifications, and combinations of the just described embodiments can be configured without departing from the scope and spirit of the system. Therefore, it is to be understood that, within the scope of the appended claims, the system may be practiced other than as specifically described herein.

What is claimed is:

1. A system for detecting an occlusion in a blood access device, the system comprising:
a blood access device in fluid communication with a calibrant solution source and a circulatory system of a patient;
a flow controller configured to sequentially draw blood from the circulatory system and flush fluid from the calibrant solution source through the blood access device, the sequential draw and flush defining a draw and flush cycle;
a sensor coupled to the blood access device and configured to generate one or more portions of a signal-time history associated with the draw and flush cycle;
a memory;
a processor; and
a computing module, stored in the memory, executable by the processor, and configured to cause the processor to:
operate the flow controller to sequentially draw blood and flush fluid through the blood access device;
determine the signal-time history for the draw and flush cycle from the sensor;
determine a time duration of a predetermined portion of the signal-time history associated with the draw and flush cycle;
determine whether the time duration is greater than a predetermined threshold value; and
detect an occlusion in the blood access device when the time duration of the signal-time history is greater than the predetermined threshold value.

2. The system of claim 1, wherein the sensor is a glucose sensor.

3. The system of claim 1, further comprising halting display of information on a display when the occlusion is detected.

4. The system of claim 1, wherein the predetermined threshold value is determined based on a standard signal-time history analyzed when an occlusion is not present.

5. A method for detecting an occlusion in a blood access device, the method comprising:
providing a blood access device in fluid communication with a calibrant solution source and a circulatory system of a patient;
providing a flow controller configured to sequentially draw blood from the circulatory system and flush fluid from the calibrant solution source through the blood access device, the sequential draw and flush defining a draw and flush cycle;
providing a sensor coupled to the blood access device and configured to generate one or more portions of a signal-time history associated with the draw and flush cycle; and
providing a processor for executing computer program code stored in a non-transitory computer-readable medium to cause the processor to:
operate the flow controller to sequentially draw blood and flush fluid through the blood access device;
determine the signal-time history for the draw and flush cycle from the sensor;
determine a time duration of a predetermined portion of the signal-time history associated with the draw and flush cycle;
determine whether the time duration is greater than a predetermined threshold value; and
detect an occlusion in the blood access device when the time duration of the signal-time history is greater than the predetermined threshold value.

6. The method of claim 5, wherein the sensor is a glucose sensor.

7. The method of claim 5, further comprising halting display of information on a display when the occlusion is detected.

8. The method of claim 5, wherein the predetermined threshold value is determined based on a standard signal-time history analyzed when an occlusion is not present.

9. A computer program product for detecting an occlusion in a blood access device, the computer program product comprising:
a non-transitory computer-readable medium comprising a set of codes for causing a computer to:
operate a flow controller to sequentially draw blood and flush fluid through a blood access device, wherein the blood access device is in fluid communication with a calibrant solution source and a circulatory system of a patient;
determine a signal-time history for the draw and flush cycle from a sensor, wherein the sensor is coupled to the blood access device;
determine a time duration of a predetermined portion of the signal-time history associated with the draw and flush cycle;
determine whether the time duration is greater than a predetermined threshold value; and
detect an occlusion in the blood access device when the time duration of the signal-time history is greater than the predetermined threshold value.

10. The computer program product of claim 9, wherein the sensor is a glucose sensor.

11. The computer program product of claim 9, further comprising halting display of information on a display when the occlusion is detected.

12. The computer program product of claim 9, wherein the predetermined threshold value is determined based on a standard signal-time history analyzed when an occlusion is not present.
